SEQUENCE LISTING (2 sequences)

SEQ ID NO: 1 - DNA, 431 bp, Rattus norvegicus
gaagtttgcgtgctgccatggagaccagagccttctggacaaccctgctgctggtcctggttgctgggtcctcctgcaaagcccaggaatttgttggcctatctccaagccaatgtatggctccaacaaatgtcagggtggactgtaactaccccactgtcacatcagagcagtgtaacaaccgtggttgctgttttgactccagcatcccaaatgtgccctggtgcttcaaacctctgcaagagacagaatgtacattttgaagctgtccaggctccaggaagggagctccacaccctggactcttgctgatggtagtggcccagggtaacactcacccctgatctgctccctcgcgccggccaatataggagctgggagtccagaagaataaagaccttacagtcagcacaaggctgttctaattgcgg

SEQ ID NO: 2 - protein, 81 aa, Rattus norvegicus
METRAFWTTLLLVLVAGSSCKAQEFVGLSPSQCMAPTNVRVDCNYPTVTSEQCNNRGCCFDSSIPNVPWCFKPLQETECTF

(19) United States
(12) Patent Application Publication
(10) Pub. No.: US 2009/0298073 A1
(43) Pub. Date: Dec. 3, 2009

(54) Kidney Toxicity Biomarkers

(76) Inventors: David L. Gerhold, Lansdale, PA (US); Daniel J. Holder, Blue Bell, PA (US); Hong Jin, Wallingford, PA (US); David J. Figueroa, Audubon, PA (US); Wendy J. Bailey, Fort Washington, PA (US); Josef S. Ozer, Souderton, PA (US); Ming Su, Maple Glen, PA (US)

Correspondence Address: MERCK AND CO., INC, P O BOX 2000, RAHWAY, NJ, 07065-0907, US

(21) Appl. No.: 12/308,918

(22) PCT Filed: Jun. 28, 2007
(86) PCT No.: PCT/US2007/015202

§ 371 Date: Dec. 29, 2008

Related U.S. Application Data
(60) Provisional application No. 60/817,727, filed on Jun. 30, 2006.
(60) Provisional application No. 60/817,752, filed on Jun. 30, 2006.

(51) Int. Cl. C12Q 1/68; C07H 21/04; C07K 14/435; C12Q 1/02; G01N 33/00

(52) U.S. Cl. 435/6; 536/23.5; 530/324; 435/29; 436/86

(57) ABSTRACT

Novel biomarkers for kidney toxicity. Said biomarkers may be useful for optimization of lead compounds, or in safety assessment.

### CROSS-REFERENCE TO RELATED APPLICATIONS

This application claims the benefit of U.S. Provisional Application Nos. 60/817,727 and 60/817,752, filed on Jun. 30, 2006, the contents of which are incorporated herein by reference in their entirety.

### FIELD OF THE INVENTION

The invention relates to novel kidney toxicity biomarkers.

### DESCRIPTION OF RELATED ART

Development of therapeutic agents to treat disease is costly and time consuming. Following the discovery of potential therapeutic compounds, the activity of these compounds must be characterized and their pharmacologic profile defined. The activity and selectivity of a given compound are crucial, as are potential safety issues, which can derail the entire process Promising therapeutic candidates must also be evaluated for all possible toxicities.

There exists a need for biomarkers for identifying early stage therapeutics that might cause kidney damage. Such biomarkers may also be used as bridging biomarkers between preclinical safety studies and clinical testing to monitor for patient safety.

### SUMMARY OF THE INVENTION

The present invention relates to novel biomarkers. More particularly, the invention relates to biomarkers for nephrotoxicity, specifically trefoil factor 3 (TFF3). In one embodiment of the present invention, there is provided a biomarker for nephrotoxicity which comprises the nucleotide sequence of SEQ ID NO: 1.

In another embodiment of the present invention, there is provided a biomarker for nephrotoxicity comprising the amino acid sequence of SEQ ID NO: 2.

The present invention further provides a method for measuring kidney toxicity, comprising the steps of measuring mRNA expression in kidney tissue of a subject and comparing said expression to a baseline level of expression, wherein a decreased level of expression correlates with nephrotoxicity.

The present invention further provides a method for measuring kidney toxicity, comprising the steps of measuring protein expression in urine of a subject, and comparing said expression to a baseline level of expression wherein a decreased level of expression correlates with nephrotoxicity.

### DETAILED DESCRIPTION OF THE INVENTION

The present invention relates to novel kidney toxicity biomarkers. More particularly, the invention relates to transcriptional biomarkers, defined as genes that are differentially expressed and in which decreases in mRNA levels indicate toxicity. Such biomarkers are useful for optimization of lead compounds, or in safety assessment for risk assessment. Ultimately, assays for these biomarkers could be useful in man to monitor clinical drug trials or to diagnose kidney disease.

The present invention further relates to novel kidney toxicity biomarkers termed “Accessible Biomarkers”, defined as proteins in blood or urine that are diagnostic for toxicity. Such biomarkers are useful for optimization of lead compounds, or in safety assessment for risk assessment. Ultimately, assays for these biomarkers could be useful in man to monitor clinical drug trials or to diagnose kidney disease.

Of particular interest is the observation that levels of trefoil factor 3 (tff3) mRNA are downregulated during nephrotoxicity. This novel kidney biomarker is identified by its cDNA sequence: gaagtttgcg tgctgccatg gagaccagag ccttctggac aaccctgctg ctggtcctgg ttgctgggtc ctcctgcaaa gcccaggaat ttgttggcct atctccaagc caatgtatgg ctccaacaaa tgtcagggtg gactgtaact accccactgt cacatcagag cagtgtaaca accgtggttg ctgttttgac tccagcatcc caaatgtgcc ctggtgcttc aaacctctgc aagagacaga atgtacat tgaagctgtc caggctccag gaagggagct ccacaccctg gactcttgct gatggtagtg gcccagggta acactcaccc ctgatctgct ccctcgcgcc ggccaatata ggagctggga gtccagaaga ataaagacct tacagtcagc acaaggctgt tctaattgcg g (SEQ ID NO: 1).

Levels of tff3 mRNA can be measured using techniques well known to those skilled in the art.

Levels of trefoil factor 3 (TFF3) protein are also downregulated during nephrotoxicity. This novel kidney biomarker is ETRAFWTTLLLVLVAGSSCKA QEFVGLSPSQCMAPTNVRVDCNYPTVTSEQCNNRGCCFDSSIPNVPWCFKPLQ ETECTF (SEQ ID NO: 2). Levels of TFF3 protein can be measured by techniques known to those skilled in the art. Examples of such techniques include antibody-based techniques such as ELISA.

### EXAMPLE 1: Measurement of transcriptional toxicity biomarkers

The biomarkers of the present invention are measured in tissues of interest during testing of a therapeutic compound. Transcriptional toxicity biomarker genes that are turned on or off in response to toxicity have been identified as follows. Approximately 20,000 rat genes were tested using microarrays in rat kidneys treated with several kidney toxicants (e.g., NaF and Bromoethylamine). RNA levels of genes in rat kidneys were assayed more sensitively and precisely using TaqMan® RT-PCR One example of the response to a kidney toxicant, Merck A, a releasable side chain carbapenem antibiotic, caused downregulation of the tff3 gene. This compound is discussed in more detail in Rosen, et al., “Reduced immunotoxicity and preservation of antibacterial activity in a releasable side-chain carbapenem antibiotic,” Science, 283 703-706, which is herein incorporated by reference in its entirety.

### EXAMPLE 2

### Measurement of toxicity biomarkers

The biomarkers of the present invention can be measured in blood or urine during testing of a novel therapeutic compound. This is especially useful in that it does not require sacrificing animals during ongoing studies. ELISA antibody assays are used to evaluate changes in protein levels.

Antibodies were purchased or made and ELISA assays were performed to measure the levels of four Kidney Toxicity Biomarker proteins in urine. The proteins are TFF3; Tarnm Horsfall protein (THP); neutrophil gelatinase-associated lipocalin (NGAL) and albumin.

Table 1 displays data from male rats treated with kidney toxicants cisplatin or gentamicin.

Data shown are averages from dose groups of 4-5 rats each. These data demonstrate that four urinary protein biomarkers reflect histopathologically-assessed kidney toxicity. Note that upon onset of nephrotoxicity, TFF3 and THP are found in lower amounts in urine, whereas NGAL and Albumin are found in higher amounts. BUN (blood urea nitrogen) and blood creatinine levels are displayed to illustrate that the TFF3 biomarker is more sensitive than these two historical biomarkers for kidney damage.

TABLE 1

| | | | | | | | | Histo- |
|---|---|---|---|---|---|---|---|---|
| | | THP | TFF3 | Albumin | NGAL | BUN | Creatinine | pathology |
| | Day | ug/mL | ng/mL | ug/mL | ug/mL | ug/mL | mg/dL | Score |
| Gentamicin | | | | | | | | |
| Dose | | | | | | | | |
| mg/Kg/Day | | | | | | | | |
| 0 | 3 | 4.3 | 868 | 0 | 12 | 16.8 | 0.44 | N |
| 20 | 3 | 4.1 | 634 | 14 | 14 | 14.8 | 0.4 | N |
| 80 | 3 | 3.4 | 280 | 221 | 13 | 14.2 | 0.4 | <1 |
| 240 | 3 | 3.2 | 132 | 129 | 17 | 18.4 | 0.42 | <1 |
| 0 | 9 | 4.4 | 591 | 0 | 14 | 14.6 | 0.4 | N |
| 20 | 9 | 4.5 | 202 | 7 | 15 | 15.2 | 0.4 | N |
| 80 | 9 | 3.1 | 121 | 190 | 14 | 15.6 | 0.5 | 1, 2 |
| 240 | 9 | 1.3 | 1.3 | 534 | 13 | 100 | 2.65 | 4 |
| 0 | 15 | 4.5 | 392 | 0 | 13 | 14.6 | 0.42 | N |
| 20 | 15 | 4.4 | 265 | 13 | 15 | 15.4 | 0.42 | <1 |
| 80 | 15 | 3.9 | 69 | 22 | 20 | 18.6 | 0.54 | 2 |
| 240 | 12 | 1.9 | 29 | 502 | 25 | ND | ND | 5 |
| Cisplatin | | | | | | | | |
| mg/Kg | | | | | | | | |
| Single Dose | | | | | | | | |
| 0 | 3 | 4.86 | 807 | 5 | 10 | 14 | 0.40 | N |
| 0.5 mk | 3 | 4.62 | 595 | 0 | 11 | 16.2 | 0.38 | N |
| 3.5 mk | 3 | 2.57 | 50 | 197 | 11 | 30.2 | 0.60 | 2 |
| 7 mk | 3 | 1.85 | 72 | 577 | 14 | 31.8 | 0.66 | 2 |
| 0 | 8 | 4.85 | 783 | 0 | 11 | 15.2 | 0.40 | N |
| 0.5 mk | 8 | 5.01 | 309 | 0 | 12 | 15 | 0.40 | N |
| 3.5 mk | 8 | 3.18 | 0.5 | 82 | 15 | 20.6 | 0.62 | 4 |
| 7 mk | 8 | 1.12 | 0.4 | 263 | 24 | 333.4615 | 5.20 | 5 |

The foregoing examples illustrate specific embodiments, but are made only by way of example and are not intended to limit the scope of this invention. Other advantages and features of this invention will become apparent from the following claims, with the scope thereof determined by reasonable equivalents, as understood by those skilled in the art.

What is claimed is:

1. A biomarker for nephrotoxicity, selected from the group consisting of the nucleotide sequence of SEQ ID NO: 1 and the amino acid sequence of SEQ ID NO: 2.

2. A method of assessing a biomarker of interest in a subject, comprising the steps of measuring MRNA expression in kidney tissue of a subject and comparing said expression to a baseline level of expression, wherein a decreased level of expression correlates with nephrotoxicity.

3. A method for measuring kidney toxicity, comprising the steps of measuring protein expression in blood or urine of a subject, and comparing said expression to a baseline level of expression wherein a decreased level of such expression correlates with nephrotoxicity.
